(19) United States
(12) Patent Application Publication
(10) Pub. No.: US 2023/0232831 A1
(43) Pub. Date: Jul. 27, 2023

(54) MONOTERPENOID AND PHENYLPROPANOID CARBONATE ESTERS AND METHODS OF THEIR MAKING AND USE AS REPELLENTS

(71) Applicant: IOWA STATE UNIVERSITY RESEARCH FOUNDATION, INC., Ames, IA (US)

(72) Inventors: James Scott KLIMAVICZ, Ames, IA (US); Joel Robert COATS, Ames, IA (US)

(21) Appl. No.: 18/015,746

(22) PCT Filed: Jul. 14, 2021
(86) PCT No.: PCT/US2021/041576

§ 371 Date: Jan. 12, 2023

Related U.S. Application Data
(60) Provisional application No. 63/051,723, filed on Jul. 14, 2020.

(51) Int. Cl. A01N 47/06; C07C 69/96; A01P 17/00

(52) U.S. Cl. CPC A01N 47/06; A01P 17/00; C07C 69/96

(57) ABSTRACT

The present application relates to monoterpenoid and phenylpropanoid carbonate esters and methods of their making and use as repellents.

This application claims the priority benefit of U.S. Provisional Pat. Application Serial No. 63/051,723, filed Jul. 14, 2020, which is hereby incorporated by reference in its entirety.

This invention was made with government support under grant number W911QY-17-1-0001 awarded by the Department of Defense. The government has certain rights in the invention.

### FIELD

The present application relates to monoterpenoid and phenylpropanoid carbonate esters and methods of their making and use as repellents.

### BACKGROUND

While significant gains have been made against malaria over the last twenty years, mosquitoes remain a threat to global health (Fernandes et al., “Revamping Mosquito-borne Disease Control to Tackle Future Threats,” Trends Parasitol. 34:359-368 (2018)). Malaria, lymphatic filariasis, dengue, yellow fever, chikungunya, West Nile fever, and other mosquito-vectored diseases are responsible for substantial human morbidity and mortality (Foster & Walker, Chapter 15 - Mosquitoes (Culicidae) In Medical and Veterinary Entomology (Third Edition), Mullen, G. R.; Durden, L. A., Eds. Academic Press: 2019; pp 261-325). While annual global malaria deaths have fallen by more than 50% since 2000, nearly four billion people remain at risk of contracting arboviruses (Benelli & Mehlhom, “Declining Malaria, Rising of Dengue and Zika Virus: Insights for Mosquito Vector Control,” Parasitol. Res. 115:1747-1754). While the use of insecticides remains an important component of global vector control strategy, the development of insecticide resistance in many populations of mosquitoes jeopardizes the advances made against these diseases (Liu, “Insecticide Resistance in Mosquitoes: Impact, Mechanisms, and Research Directions,” Annu. Rev. Entomol. 60:537-559 (2015); Weill et al., “Insecticide Resistance in Mosquito Vectors,” Nature 423:136-137 (2003); Hemingway et al., “The Molecular Basis of Insecticide Resistance in Mosquitoes,” Insect Biochem. Mol. Biol. 34:653-665 (2004)). The use of insect repellents is an important part of integrated pest management strategies, and is complementary to pesticide use (Axtell, “Principles of Integrated Pest Management (IPM) in Relation to Mosquito Control,” Mosq. News 39:709-718 (1979); Metcalf & Metcalf, “Attractants, Repellents, and Genetic Control in Pest Management,” In Introduction to Insect Pest Management, John Wiley New York: 1994; pp 355-356; Peterson & Coats, “Insect Repellents - Past, Present and Future,” Pestic. Outlook 12:154-158 (2001)). In particular, spatial repellents are of interest for the exclusion of insects from entering spaces occupied by human hosts and are complementary to other vector control methods like insecticide-treated mosquito nets and insecticide residual spraying (Achee et al., “Spatial Repellents: From Discovery and Development to Evidence-based Validation,” Malar. J. 11:164 (2012)).

Amongst the best-known arthropod repellents are N,N-diethyl-m-toluamide (DEET), picaridin, IR3535, and p-menthane-3,8-diol (PMD) (Katz et al., “Insect Repellents: Historical Perspectives and New Developments,” J. Am. Acad. Dermatol. 58:865-871 (2008)). However, while these compounds provide protection from mosquito bites, they only do so through direct contact with the insect, as opposed to deterring insects from entering a volume of space (Peterson & Coats, “Insect Repellents - Past, Present and Future,” Pestic. Outlook 12:154-158 (2001); Achee et al., “Spatial Repellents: From Discovery and Development to Evidence-based Validation,” Malar. J. 11:164 (2012)). In contrast, plant essential oils have long been used as insecticides and insect repellents (Isman, “Plant Essential Oils for Pest and Disease Management,” Crop Protect. 19:603-608 (2000)), and their complex compositions frequently contain volatile compounds that potentially lend themselves well for use as spatial repellents. These oils are frequently composed of mono- and sesquiterpenoid and phenylpropanoid hydrocarbons, alcohols, aldehydes, ketones, and esters, many of which are repellent or insecticidal (Nakatsu et al., “Biological Activity of Essential Oils and Their Constituents,” In Stud. Nat. Prod. Chem., Atta ur, R., Ed. Elsevier: 2000; Vol. 21, pp 571-631). While readily available, these compounds are often not ideal for use as insect repellents due to their strong scents, high volatility and corresponding short duration of protection, and concerns regarding skin sensitivity (Barnard, D. R., “Repellency of Essential Oils to Mosquitoes (Diptera: Culicidae),” J. Med. Entomol. 36:625-629 (1999); de Groot & Frosch, “Adverse Reactions to Fragrances,” Contact Dermatitis 36:57-86 (1997); Nerio et al., “Repellent Activity of Essential Oils: A Review,” Bioresour. Technol. 101:372-378 (2010)).

Plant terpenoids are attractive starting points for the development of novel insect repellents as minor changes in structure can trigger significant changes in the potency of the compound, though it is normally not obvious how these changes will affect efficacy. A common strategy is to modify one or more functional groups in the parent terpenoid while leaving the bulk of the molecule intact. Methyl ethers of some monoterpenoids, such as the naturally-occurring methyl ethers of thymol and carvacrol, are less potent as repellents than the parent monoterpenoids (Tabanca et al., “Bioassay-guided Investigation of Two Monarda Essential Oils as Repellents of Yellow Fever Mosquito Aedes aegypti,” J. Agric. Food Chem. 61:8573-8580 (2013); Tabanca et al., “Eupatorium capillifolium Essential Oil: Chemical Composition, Antifungal Activity, and Insecticidal Activity,” Nat. Prod. Commun. 5:1934578X1000500913 (2010)). Terpenoid alcohols have been esterified to make repellent or insecticidal compounds. In many cases, these esters had improved physical properties, such as lower volatility and improved long-term spatial repellency, while others were better fumigants or contact insecticides (Klimavicz et al., “Monoterpenoid Isovalerate Esters as Long-lasting Spatial Mosquito Repellents,” In Advances in the Biorational Control of Medical and Veterinary Pests, American Chemical Society: 2018; Vol. 1289, pp 205-217; Devappa et al., “Potential of Using Phorbol Esters as an Insecticide Against Spodoptera frugiperda,” Ind. Crops Prod. 38:50-53 (2012); Moore, “Esters as Repellents,” J. N. Y. Entomol. Soc. 42:185-192 (1934); and Rice & Coats, “Insecticidal Properties of Several Monoterpenoids to the House Fly (Diptera: Muscidae), Red Flour Beetle (Coleoptera: Tenebrionidae), and Southern Corn Rootworm (Coleoptera: Chrysomelidae),” J. Econ. Entomol. 87:1172-1179 (1994)). Monoterpenoid esters using glycine and γ-aminobutyric acid (GABA) residues have also been developed (Nesterkina et al., “Repellent Activity of Monoterpenoid Esters with Neurotransmitter Amino Acids Against Yellow Fever Mosquito, Aedes aegypti,” Open Chem. 16:95 (2018)). These amino acids were chosen both because of their role as neurotransmitters and because many monoterpenoids have been found to be allosteric modulators of insect GABA receptors (Tong, “Investigation of Mechanisms of Action of Monoterpenoid Insecticides on Insect Gamma-aminobutyric Acid Receptors and Nicotinic Acetylcholine Receptors,” Ph.D. Thesis, Iowa State University, Ames, IA, 2010). Although these amino esters had higher minimum effective doses for mosquito repellency than the parent monoterpenoids, they also slowly hydrolyze to release free monoterpenoids, which may provide for the extended release of repellent monoterpenoids.

Callicarpenal, a sesquiterpenoid found in Callicarpa spp. (Verbenaceae) has previously been shown to be repellent to mosquitoes (Cantrell et al., “Isolation and Identification of Mosquito Bite Deterrent Terpenoids from Leaves of American (Callicarpa americana) and Japanese (Callicarpa japonica) Beautyberry,” J. Agric. Food Chem. 53:5948-5953 (2005)), and an epoxide derivative of this natural product was significantly more toxic to mosquitoes than the parent compound (Cantrell et al., “Structure-activity Relationship Studies on the Mosquito Toxicity and Biting Deterrency of Callicarpenal Derivatives,” Chem. Biodivers. 6:447-458 (2009)). Likewise, many synthetic derivatives of the related terpenoids valencene and nootkatone were more repellent than nootkatone itself against the Formosan termite Coptotermes formosanus (Isoptera: Rhinotermitidae) (Zhu et al., “Structural Requirements for Repellency: Norsesquiterpenes and Sesquiterpenoid Derivatives of Nootkatone Against the Formosan Subterranean Termite (Isoptera: Rhinotermitidae),” Pest Manage. Sci. 66:875-878 (2010)). Lactams derived from the natural monoterpenoid nepetalactone, a repellent component of catnip, Nepeta cataria (Lamiaceae), essential oil (Peterson & Coats, “Catnip Essential Oil and its Nepetalactone Isomers as Repellents for Mosquitoes,” In Recent Developments in Invertebrate Repellents, American Chemical Society: 2011; Vol. 1090, pp 59-65), have also been synthesized as effective mosquito feeding deterrents (Chauhan et al., “Biobased Lactams as Novel Arthropod Repellents,” Nat. Prod. Commun. 9:1934578X1400901201 (2014)).

Despite the push toward a greater exploration of the chemical space around spatially repellent molecules, surprisingly little work has been done to investigate carbonate esters as potential repellents. 4-Cycloocten-1-yl methyl carbonate, an artificial fragrance ingredient, has been patented as a repellent for some dipterans, including mosquitoes, house flies, and horn flies (U.S. Pat. No. 5,417,009 to Butler et al.). Asymmetric carbonates of 1,3-dihydroxyacetone were also synthesized under the premise that these compounds might slowly hydrolyze to release repellent compounds (Garson & Garner, “Unsymmetrical Carbonates as Potential Long-lasting Insect Repellents,” J. Pharm. Sci. 60:1083-1085 (1971)), including 2-ethyl-1,3-hexanediol (Rutgers 612), a repellent that has fallen out of favor due to possible endocrine disruption after repeated exposure (Neeper-Bradley et al., “Evaluation of the Developmental Toxicity Potential of 2-ethyl-1, 3-hexanediol in the Rat by Cutaneous Application,” J. Toxicol.: Cutaneous Ocular Toxicol. 13:203-214 (1994); Brown & Hebert, “Insect Repellents: An Overview,” J. Am. Acad. Dermatol. 36:243-249 (1997)). Menthol propylene glycol carbonate, an artificial flavoring compound derived from the monoterpenoid menthol, has also been found to be an effective mosquito repellent (Kweka et al., “Protective Efficacy of Menthol Propylene Glycol Carbonate Compared to N,N-diethyl-methylbenzamide Against Mosquito Bites in Northern Tanzania,” Parasites Vectors 5:189 (2012); U.S. Pat. Application Serial No. 10/595,143 to Matias). An examination of monoterpenoid carbonates was warranted to assess the utility of this functional group for future development of novel repellent compounds.

The present application is directed to overcoming deficiencies in the art.

### SUMMARY

One aspect of the present application relates to a compound of formula (I) having the following structure:

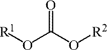

or a stereoisomer, salt, oxide, or solvate thereof, wherein
R¹ is a monoterpenoid or phenylpropanoid moiety;
R² is selected from C₁-C₁₀ alkyl optionally substituted with halogen, C₂-C₁₀ alkenyl, phenyl, and —(CH₂)ₙ—phenyl; and
n is an integer from 0-3.

Another aspect of the present application relates to a composition comprising a carrier; and a compound of formula (I) having the following structure:

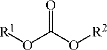

or a stereoisomer, salt, oxide, or solvate thereof, wherein
R¹ is a monoterpenoid or phenylpropanoid moiety;
R² is selected from C₁-C₁₀ alkyl optionally substituted with halogen, C₂-C₁₀ alkenyl, phenyl, and —(CH₂)ₙ—phenyl; and
n is an integer from 0-3.

A further aspect of the present application relates to a method of repelling a pest. This method involves applying to a target area a composition comprising a compound of formula (I) having the following structure:

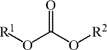

or a stereoisomer, salt, oxide, or solvate thereof, wherein
R¹ is a monoterpenoid or phenylpropanoid moiety;
R² is selected from C₁-C₁₀ alkyl optionally substituted with halogen, C₂-C₁₀ alkenyl, phenyl, and —(CH₂)ₙ—phenyl; and
n is an integer from 0-3;
wherein said applying is carried out under conditions effective to repel a pest.

A series of citronellyl carbonates were readily synthesized from chloroformates and citronellol, with the most effective spatial repellent, citronellyl ethyl carbonate, repelling approximately 90% of mosquitoes in a short-term repellency assay extending out to 2.5 hours. Given the success of citronellyl ethyl carbonate, several other monoterpenoids were used to produce ethyl carbonates, most of which provided spatial repellency at or above 90% over the course of the bioassay.

The volatile monoterpenoid carbonate esters described herein constitute a new class of spatial repellents that are similar to, but chemically distinct from, monoterpenoid carboxylate esters that have also been shown to provide good spatial repellency. As many of these carbonates have shown excellent repellency in the short-term bioassay, these compounds will be explored as long-term spatial repellents, while expanding the number of parent monoterpenoids used to synthesize these compounds.

### BRIEF DESCRIPTION OF THE DRAWINGS

FIG. 1 is a graph showing short-term repellency of citronellyl carbonates against Culex pipiens.

FIG. 2 is a graph showing short-term repellency of isobutyl thymyl carbonate, 2d, against methyl thymyl carbonate (2a) and citronellyl isobutyl carbonate (1d).

FIG. 3 is a graph showing short-term repellency of monoterpenoid ethyl carbonates against Culex pipiens.

FIG. 4 is a graph showing the repellency observed when 1 mL of a 0.5% solution of each compound was applied to a filter paper, dried for 15 minutes, and placed at the treated end of the repellency chamber.

FIG. 5 is a graph showing the repellency observed when 1 mL of each compound was applied to a filter paper placed at the treated end of the repellency chamber 5 hours after compound was applied.

FIG. 6 is a graph showing repellency of various compounds against the mosquito species Culex pipiens observed when 1 mL of a 0.5% solution of each compound was applied to a filter paper, dried for 15 minutes, and placed at the treated end of the repellency chamber.

FIG. 7 is a graph showing repellency of various compounds against the mosquito species Aedes aegypti observed when 1 mL of a 0.5% solution of each compound was applied to a filter paper, dried for 15 minutes, and placed at the treated end of the repellency chamber.

FIG. 8 is a graph showing the repellency of various compounds observed when 1 mL of each compound was applied to a filter paper placed at the treated end of the repellency chamber 5 hours after compound was applied.

### DETAILED DESCRIPTION

The present application relates to compounds, including insect repellent compounds, compositions containing the compounds, and methods of making and using the compounds. In particular, the present application relates to monoterpenoid and phenylpropanoid compounds derived from biorational sources for use against arthropods. As discussed in more detail infra, the monoterpenoid and phenylpropanoid derivative compounds of the present application are particularly suited for use as repellents against various mosquito species.

In discussing the compounds of the present application described herein, the following terms are provided for clarity.

As used herein, the term “monoterpenoid” refers to a monoterpene-like substance and may be used loosely herein to refer collectively to monoterpenoid derivatives as well as monoterpenoid analogs. By the term “monoterpene,” it is meant a compound having a 10-carbon skeleton with non-linear branches. A monoterpene technically refers to a compound with two isoprene units connected in a head-to-end manner. Monoterpenoids can therefore include monoterpenes, alcohols, ketones, aldehydes, esters, ethers, acids, hydrocarbons without an oxygen functional group, and so forth. It is common practice to refer to certain phenolic compounds, such as eugenol, thymol, and carvacrol, as monoterpenoids because their function is essentially the same as a monoterpenoid. However, these compounds are not technically “monoterpenoids” (or “monoterpenes”) because they are not synthesized by the same isoprene biosynthesis pathway, but rather by production of phenols from tyrosine. However, common practice will be followed herein.

The term “phenylpropanoid” refers to a diverse group of organic compounds that are synthesized by plants from the amino acid phenylalanine. Their name is derived from the six-carbon, aromatic phenyl group and the three-carbon propene tail of cinnamic acid, which is synthesized from phenylalanine in the first step of phenylpropanoid biosynthesis. Phenylpropanoids are found throughout the plant kingdom, where they serve as essential components of a number of structural polymers, provide protection from ultraviolet light, defend against herbivores and pathogens, and mediate plant-pollinator interactions as floral pigments and scent compounds.

According to one embodiment, the monoterpenoid or phenylpropanoid of the compounds of the present application is derived from a biorational source, such as a plant volatile or as a constituent of plant essential oils obtained from the leaf tissue, stem tissue, root tissue, or mixture thereof. In another embodiment, the monoterpenoid or phenylpropanoid used for synthesis to obtain a higher molecular weight, higher polarity, or decreased volatility is obtained from a synthetic source. The term “volatility” as used herein is defined as the property of a substance having a low boiling point and a high vapor pressure at ordinary temperatures and pressures. Similarly, the term “volatile” is considered to refer to a compound that is readily vaporizable at a relatively low temperature. A “slightly volatile” compound may be considered to have a vapor pressure of between about 0.05 Pascal (Pa) and two (2) Pa. Slightly volatile repellents can be considered to include DEET (vapor pressure of 0.22 Pa), as well as many of the repellent compounds of the present application. “Slightly volatile” is a desirable property for a repellent because it provides an additional route of exposure against a target pest, i.e., fumigation, as discussed infra. Furthermore, the same amount of such a repellent is effective over a larger target area as compared with a non-volatile repellent, which is limited to only a contact route of exposure. “High volatility” is generally considered an undesirable property for a repellent, because such repellents typically dissipate too rapidly to be effective. Citronella is a repellent with high volatility. The essential oil of a plant is considered to include only “volatile” components. Similarly, the term “plant volatile” as used herein refers to a volatilizing compound from any part of a plant, including, but not limited to, a leaf, root, flower or flower bud, fruit, vegetable, stem, and so forth.

As used herein, the term “alkyl” means an aliphatic hydrocarbon group which may be straight or branched. When not otherwise restricted, the term refers to an alkyl of from 1 to 10 carbons. Exemplary alkyl groups include ethyl, n-propyl, i-propyl, n-butyl, t-butyl, n-pentyl, 3-pentyl, and the like.

The term “alkenyl” means an aliphatic hydrocarbon group containing a carbon— carbon double bond and which may be straight or branched having from 2 to about 10 carbon atoms in the chain. Exemplary alkenyl groups include ethenyl, propenyl, n-butenyl, isoprene, and i-butenyl. The term “alkenyl” may also refer to a hydrocarbon chain having 2 to 10 carbons containing at least one double bond and at least one triple bond.

The term “halogen” as used herein is intended to include fluorine, bromine, chlorine, and iodine while the term “halide” is intended to include fluoride, bromide, chloride, and iodide anion.

The term “substituted” specifically envisions and allows for one or more substitutions that are common in the art. However, it is generally understood by those skilled in the art that the substituents should be selected so as to not adversely affect the useful characteristics of the compound or adversely interfere with its function. According to one embodiment, the compounds of the present application are unsubstituted. “Unsubstituted” atoms bear all of the hydrogen atoms dictated by their valency.

According to another embodiment, the compounds of the present application are substituted. By “substituted” it is meant that a group may have a substituent at each substitutable atom of the group (including more than one substituent on a single atom), provided that the designated atom’s normal valency is not exceeded and the identity of each substituent is independent of the others. For example, up to three H atoms in each residue are replaced with substituents such as halogen, haloalkyl, hydroxy, lower alkoxy, carboxy, carboalkoxy (also referred to as alkoxycarbonyl), carboxamido (also referred to as alkylaminocarbonyl), cyano, nitro, amino, alkylamino, dialkylamino, mercapto, alkylthio, sulfoxide, sulfone, acylamino, amidino, phenyl, benzyl, heteroaryl, phenoxy, benzyloxy, or heteroaryloxy. When a substituent is keto (i.e., =0), then two hydrogens on the atom are replaced. Combinations of substituents and/or variables are permissible only if such combinations result in stable compounds; by “stable compound” it is meant a compound that is sufficiently robust to survive isolation to a useful degree of purity from a reaction mixture, and formulation into an agent intended for a suitable use.

The term “compound,” and equivalent expressions, are meant to embrace compounds of the present application described herein. Also contemplated are salts, oxides, solvates, e.g., hydrates, and inclusion complexes of the compounds, where the context so permits, as well as any stereoisomeric form, or a mixture of any such forms of that compound in any ratio. Inclusion complexes are described in Remington, The Science and Practice of Pharmacy, 19th Ed. 1:176-177 (1995), which is hereby incorporated by reference in its entirety. The most commonly employed inclusion complexes are those with cyclodextrins, and all cyclodextrin complexes, natural and synthetic, are specifically encompassed within the claims.

Compounds described herein may contain one or more asymmetric centers and may thus give rise to enantiomers, diastereomers, and other stereoisomeric forms. Each chiral center may be defined, in terms of absolute stereochemistry, as (R)- or (S)—. This technology is meant to include all such possible isomers, as well as mixtures thereof, including racemic and optically pure forms. Optically active (R)- and (S)—, (-)- and (+)-, or (D)- and (L)- isomers may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers. Likewise, all tautomeric forms are also intended to be included.

One aspect of the present application relates to a compound of formula (I) having the following structure:

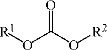

or a stereoisomer, salt, oxide, or solvate thereof, wherein
R¹ is a monoterpenoid or phenylpropanoid moiety;
R² is selected from C₁-C₁₀ alkyl optionally substituted with halogen, C₂-C₁₀ alkenyl, phenyl, and —(CH₂)ₙ—phenyl; and
n is an integer from 0-3.

In one embodiment of the compound of formula (I) described herein, the following provisos apply:
when R² is C₁ alkyl, C₂ alkyl, t-butyl, phenyl, or C₃ alkenyl, R¹ is not

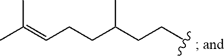

when R² is C₂ alkyl, R¹ is not

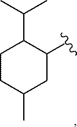

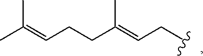

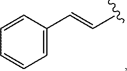

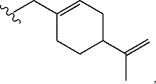

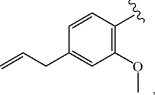

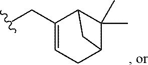

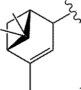

These provisos may apply to compounds of formula (I), compositions comprising compounds of formula (I), and methods of repelling pests using compounds and/or compositions of formula (I).

In one embodiment of the compound of formula (I), R¹ is a monoterpenoid moiety.

In one embodiment of the compound of formula (I), the monoterpenoid moiety is selected from the group consisting of

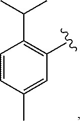

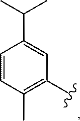

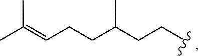

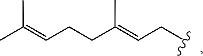

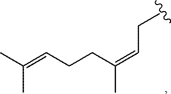

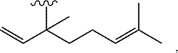

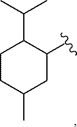

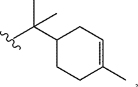

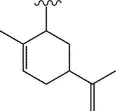

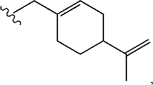

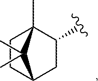

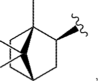

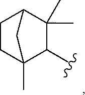

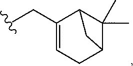

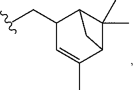

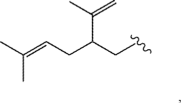

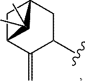

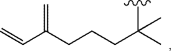

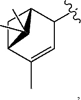

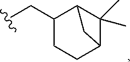

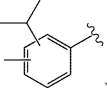

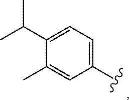

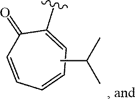

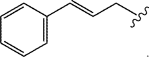

In one embodiment of the compound of formula (I), R¹ is a phenylpropanoid moiety.

In one embodiment of the compound of formula (I), the phenylpropanoid moiety is selected from the group consisting of

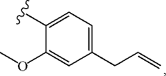

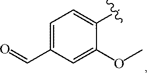

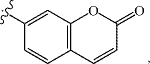

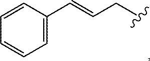

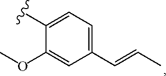

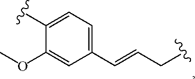

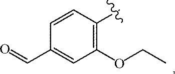

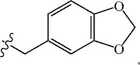

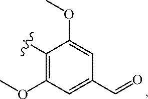

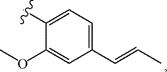

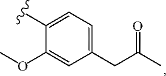

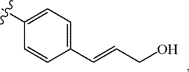

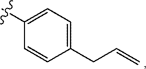

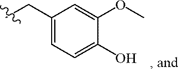

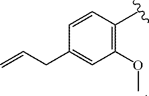

In one embodiment of the compound of formula (I), R² is C₁-C₁₀ alkyl. In particular embodiments, the compound of formula (I) has a structure of

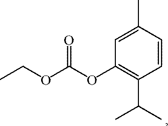

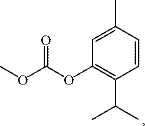

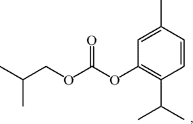

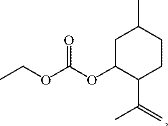

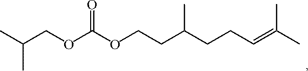

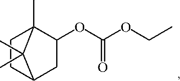

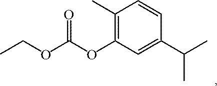

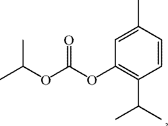

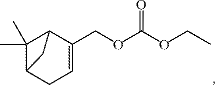

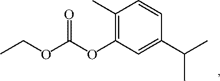

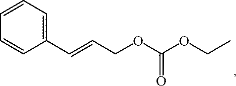

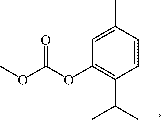

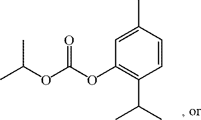

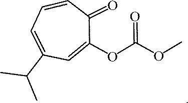

In one embodiment of the compound of formula (I), R² is C₁-C₁₀ alkyl substituted with halogen. In a particular embodiment, the compound of formula (I) has a structure

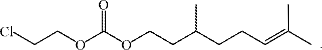

In one embodiment of the compound of formula (I), R² is —(CH₂)ₙ—phenyl. In a particular embodiment, the compound of formula (I) has the following structure:

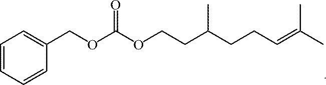

Compounds of formula (I) can be made using synthesis procedures described in the Examples below.

Another aspect of the present application relates to a composition comprising a carrier and a compound of formula (I) having the following structure:

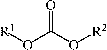

or a stereoisomer, salt, oxide, or solvate thereof, wherein
R¹ is a monoterpenoid or phenylpropanoid moiety;
R² is selected from C₁-C₁₀ alkyl optionally substituted with halogen, C₂-C₁₀ alkenyl, phenyl, and —(CH₂)ₙ—phenyl; and
n is an integer from 0-3.

In one embodiment of the composition of the present application, the compound is in a substantially pure form.

In one embodiment, the compound is a single enantiomer or diastereomer.

In one embodiment, the compound is in a racemic or diastereomeric mixture.

In one embodiment, the carrier is selected from a solid, liquid, and gas.

In one embodiment, the composition is in the form of a lotion, spray, or cream.

In one embodiment, the composition further comprises a fragrance, perfume, or cologne.

In one embodiment, the composition of the present application includes one or more compounds of formula (I) formulated according to any of the embodiments described herein.

The compounds of the present application can be used in undiluted or diluted form and can be converted into formulations or compositions customary for repellents. They can be used in all the presentation forms customary in cosmetics, including, without limitation, in the form of solutions, emulsions, gels, ointments, pastes, creams, powders, sticks, sprays, aerosols, and fumigants.

Thus, another aspect of the present application relates to a composition (or formulation) comprising a compound of the present application (as described herein) and a carrier.

For use in the non-cosmetic sector, compounds of formula (I) can be incorporated, for example, into granules, oily spraying agents, or slow release formulations. Such formulations are prepared in a known manner by mixing or diluting the compounds of formula (I) with one or more solvents (e.g., xylene, chlorobenzenes, paraffins, methanol, ethanol, isopropanol, or water), carriers (e.g., kaolins, aluminas, talc, chalk, highly disperse silicic acid and silicates, nanoclays), emulsifying agents (e.g., polyoxyethylene fatty acid esters, polyoxyethylene fatty alcohol ethers, alkylsulphonates and arylsulphonates), and dispersing agents (e.g., lignin, sulphite waste liquors and methylcellulose), any of which are considered “carriers” for purposes of the compositions of the present application.

The compounds of the present application can be mixed with one another in the formulations to form the compositions or can also be used as mixtures with other known active compounds (e.g., sunscreen agents). The compositions in general contain between about 0.1 and about 95% (e.g., 0.1-95%) by weight of active compound, or between about 0.5 and about 90% (e.g., 0.5-90%).

In one embodiment, the composition is in the form of a lotion, spray, or cream. In another embodiment, the composition further includes a fragrance, perfume, or cologne.

In one embodiment, the composition of the present application is formulated to be administered by topical application to the skin (i.e., keratinous tissue). Accordingly, the composition preferably has good dermatological and aesthetic properties and will not cause any safety or toxicity concerns.

The carrier used in this and other compositions of the present application can be in a wide variety of forms, including emulsion carriers, such as oil-in-water, water-in-oil, and oil-in-water-in-silicone emulsions, creams, ointments, ophthalmic ointments, aqueous solution, lotions, gels, or aerosols. As will be understood by the skilled artisan, a given component will distribute primarily into either the water or oil/silicone phase, depending upon the water solubility/dispersibility of the component in question. A safe and effective amount of carrier is from about 50% to about 99.99%, from about 80% to about 99.99%, from about 90% to about 98%, or from about 90% to about 95% of the composition.

Emulsions generally contain an effective amount of a compound of the present application and a lipid or oil. Lipids and oils may be derived from animals, plants, or petroleum, and can be natural or synthetic. Emulsions may also contain a humectant such as glycerin. Emulsions may further contain from about 1% to about 10% or from about 2% to about 5%, of an emulsifier, based on the weight of the carriers. Emulsifiers may be ionic, anionic, or cationic. The emulsion may also contain an anti-foaming agent to minimize foaming upon application to the keratinous tissue. Anti-foaming agents include high molecular weight silicones and other materials well known in the art for such use.

Suitable emulsions may have a wide range of viscosities, depending upon the product form. Exemplary low viscosity emulsions have a viscosity of about 50 centistokes or less, about 10 centistokes or less, or about 5 centistokes or less. The emulsion may also contain anti-foaming agents to minimize foaming upon application to the skin.

Other carriers include oil-in-water emulsions having a continuous aqueous phase and a hydrophobic, water-insoluble phase dispersed therein. Oil-in-water emulsions may comprise from about 25% to about 98%, from about 65% to about 95%, or from about 70% to about 90% water by weight of the carrier.

The hydrophobic phase is dispersed in the continuous aqueous phase. The hydrophobic phase may contain water insoluble or partially soluble materials such as are known in the art including, but not limited to, silicones. The compositions of the present application include, but are not limited to, lotions and creams, and may comprise a dermatologically acceptable emollient. As used herein, “emollient” refers to a material useful for preventing or relieving dryness, as well as for protecting the skin. A wide variety of suitable emollients is known and any may be used with the compositions of the present application. Numerous examples of materials suitable for use as an emollient are provided in Sagarin, Cosmetics, Science, and Technology 2nd Edition Vol. 1, pp 3243 (1972), which is hereby incorporated by reference in its entirety. One specific emollient is glycerin. Glycerin may be used in an amount of from about 0.001% to about 20%, from about 0.01% to about 10%, or from about 0.1% to about 5% w/w of the total composition.

Lotions and creams generally comprise a solution carrier system and one or more emollients. Lotions typically comprise from about 1% to about 20% or from about 5% to about 20% of emollient; from about 50% to about 90% or from about 60% to about 80% water; and an effective amount of a compound of the present application.

Ointments may comprise a simple carrier base of animal or vegetable oil or semisolid water-soluble carriers. Ointments may further comprise a thickening agent and/or an emollient. For example, an ointment may comprise from about 2% to about 20% of an emollient, about 0.1 to about 2% of a thickening agent, and an effective amount of a compound of the present application.

Compositions of the present application may also include optional components, which should be suitable for application to keratinous tissue, i.e., when incorporated into the composition they are suitable for use in contact with human keratinous tissue without undue toxicity, incompatibility, instability, allergic response, and the like within the scope of sound medical judgment. In addition, such optional components are useful provided that they do not unacceptably alter the benefits of the active compounds of the present application. The CTFA Cosmetic Ingredient Handbook, Second Edition (1992) (which is hereby incorporated by reference in its entirety), describes a wide variety of non-limiting cosmetic and pharmaceutical ingredients commonly used in the skin care industry, which are suitable for use in the compositions of the present application. Examples of these ingredient classes include abrasives, absorbents, aesthetic components such as fragrances, pigments, colorings, essential oils, skin sensates, astringents (e.g., clove oil, menthol, camphor, eucalyptus oil, eugenol, menthyl lactate, hazel distillate), anti-acne agents, anti-caking agents, antifoaming agents, antimicrobial agents, antioxidants, binders, biological additives, buffering agents, bulking agents, chelating agents, chemical additives, colorants, cosmetic astringents cosmetic biocides, denaturants, drug astringents, external analgesics, film formers or materials such as polymers for aiding the film-forming properties and substantivity of the composition (e.g., copolymer of eicosene and vinyl pyrrolidone), opacifying agents, pH adjusters, propellants, reducing agents, sequestrants and/or healing agents (e.g., panthenol and derivatives such as ethyl panthenol), aloe vera, pantothenic acid and its derivatives, allantoin, and bisabolol), skin treating agents, thickeners, and vitamins and derivatives thereof.

The compounds and compositions of the present application repel insects.

With respect to the compositions containing the compounds of the present application, the appropriate dose regimen, the amount of each dose administered, and specific intervals between doses of the active compound may depend upon the particular active compound employed, the age and condition of the subject to which the compositions is administered (if, in fact, it is intended to be administered, e.g., as a topical application to a subject), and the desired repellent effect.

As one skilled in the art will readily appreciate, the compounds of the present application can be used alone or in combination with one another, as well as in combination with the other insect repellents (e.g., those currently commercially available, some of which are described herein).

The compositions of the present application may be useful for cosmetic purposes. Cosmetic applications include the topical application of compositions containing one or more compounds of the present application.

An effective dosage and treatment protocol can be determined by conventional means, starting with a low dose in laboratory animals, and then increasing the dosage while monitoring the effects, and systematically varying the dosage regimen as well.

Compositions of the present application may be administered by topical application. For topical administration, the compounds of the present application can be formulated as a foam or mousse, solution, gel, lotion, ointment, cream, suspension, paste, liniment, powder, tincture, aerosol, transdermal drug delivery system, or the like, in a pharmaceutically or cosmetically acceptable form by methods well known in the art. The composition can be in any variety of forms common in the pharmaceutical or domestic arts for topical application to animals or humans, including solutions lotions, sprays, creams, ointments, salves, gels, aerosols, etc., as set forth above. In some embodiments, suitable agents are those that are viscous enough to remain on the treated area, those that do not readily evaporate, and/or those that are easily removed by rinsing with water topically with the aid of soaps, cleansers, and/or shampoos. Actual methods for preparing topical formulations are known or apparent to those skilled in the art.

The compounds of the present application are less volatile than naturally occurring monoterpenoids, and more closely match the volatility and MW of sesquiterpenoids, meaning a compound having a 15-carbon scaffold with non-linear branches. The term is often used loosely to refer collectively to sesquiterpenoid derivatives as well as sesquiterpenoid analogs. Sesquiterpenoids can include sesquiterpenes, alcohols, ketones, aldehydes, ethers, acids, hydrocarbons without an oxygen functional group, and so forth.

For protection from arthropods such as blood-sucking insects or mites, the compounds and/or compositions of the present application are generally either applied to human or animal skin, or items of clothing and other objects are treated with the compounds. The compounds may be dispensed into the environment (e.g., outdoors or indoors) in vapor form (e.g., an aerosol).

The compounds of the present application, when combined with a suitable carrier or vehicle, are useful as insect repellents. Target areas for such use include, without limitation, people, pets, livestock, cupboards, containers, houses, yards, gardens, and so forth. Thus, target areas can include inanimate objects in the vicinity of a target area, including but not limited to, plants, articles of clothing, premises, tents, pillows, bed nets, blankets, automobiles, etc.

The repellents can be used against a variety of target pests including, without limitation, blood-sucking insects, biting insects, cockroaches, mosquitoes, blackfly, fleas, house flies, barn fly, face fly, bush fly, deer fly, horse fly, gnats, beetle, beer bug, louse, bed bug, earwig, ant, aphid, spruce bud worm, corn borer, sand flea, tsetse fly, assassin bug, biting flies, sand fly, stored grain pests (e.g., maize weevil, red flour beetle, saw-toothed grain beetle, Indian meal moth), clothes moths, ticks, mites, spiders, phytophagous pests, hematophagous pests, and other arthropod pests.

Different formulations or routes of exposure can provide for even further uses. For example, in addition to exposing the target pest to the repellent by contact, and possibly aquatic exposure, any of the novel repellents described herein can also be used as fumigants. Useful amounts to evoke repellency (“repellent” amounts) will depend on the particular application technique used and on the specific conditions in the area at the time of application. Such amounts can readily be determined by those skilled in the art.

A further aspect of the present application relates to a method of repelling a pest. This method involves applying to a target area a composition comprising a compound of formula (I) having the following structure:

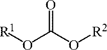

or a stereoisomer, salt, oxide, or solvate thereof, wherein
R¹ is a monoterpenoid or phenylpropanoid moiety;
R² is selected from C₁-C₁₀ alkyl optionally substituted with halogen, C₂-C₁₀ alkenyl, phenyl, and —(CH₂)ₙ—phenyl; and
n is an integer from 0-3;
wherein said applying is carried out under conditions effective to repel a pest.

In one embodiment of this method, in the compound of formula (I), R¹ is a monoterpenoid moiety.

In one embodiment of this method, in the compound of formula (I), the monoterpenoid moiety is selected from the group consisting of

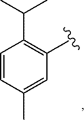

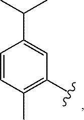

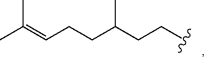

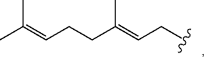

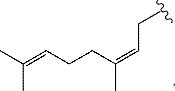

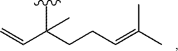

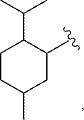

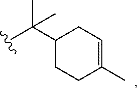

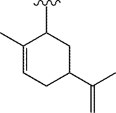

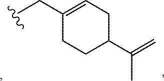

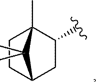

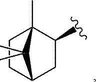

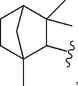

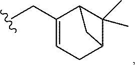

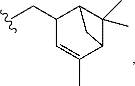

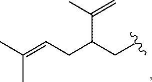

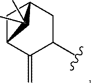

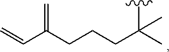

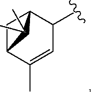

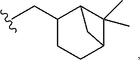

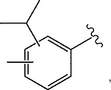

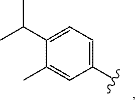

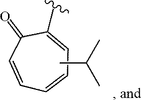

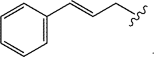

In one embodiment of this method, in the compound of formula (I), R¹ is a phenylpropanoid moiety.

In one embodiment of this method, in the compound of formula (I), the phenylpropanoid moiety is selected from the group consisting of

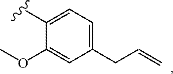

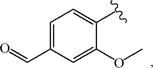

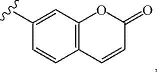

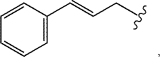

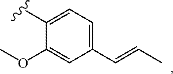

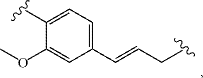

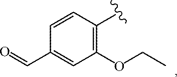

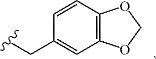

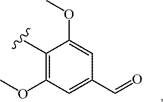

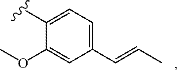

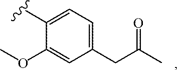

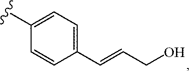

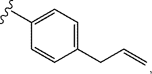

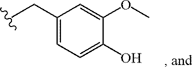

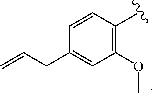

In one embodiment of this method, in the compound of formula (I), R² is C₁-C₁₀ alkyl.

In one embodiment of this method, in the compound of formula (I), is selected from the group consisting of

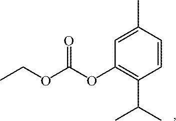

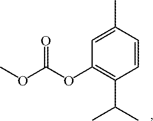

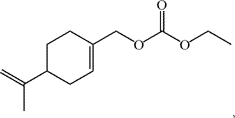

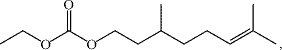

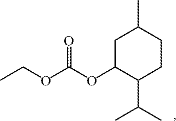

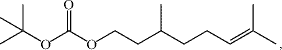

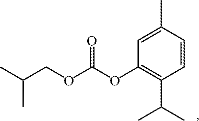

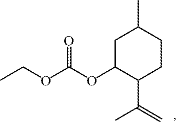

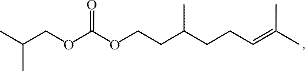

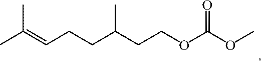

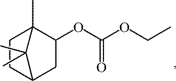

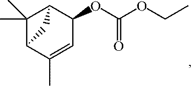

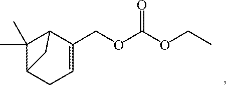

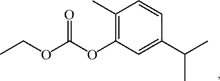

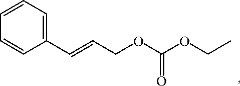

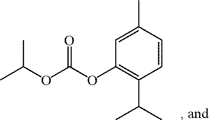

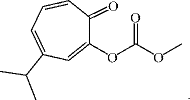

In one embodiment of this method, in the compound of formula (I), R² is C₁-C₁₀ alkyl substituted with halogen. In a particular embodiment, the compound of formula (I) has the following structure:

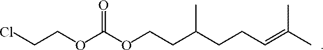

In one embodiment of this method, in the compound of formula (I), R² is C₂-C₁₀ alkenyl. In a particular embodiment, the compound of formula (I) has the following structure:

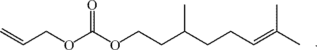

In one embodiment of this method, in the compound of formula (I), R² is phenyl. In a particular embodiment, the compound of formula (I) has the structure:

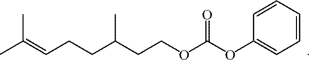

In one embodiment of this method, in the compound of formula (I), R² is —(CH₂)ₙ—phenyl. In a particular embodiment, the compound of formula (I) has the structure:

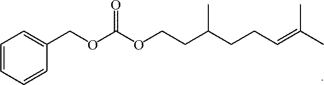

In one embodiment of the method of the present application, said applying is carried out with a vapor delivery system. This refers to vapor delivery systems that are based on passive flow control nozzles that utilize permeable polymeric membranes. There are two primary preferred systems or approaches: (i) fixed supply, stand-alone units and (ii) replenished, distributed systems (such as replenished by gravity or by pumps). The vapor delivery system with a fixed supply is used to deliver volatile compounds in either open local environments or open field environments. On the other hand, the pumped delivery system with a piped supply distribution header is uniquely suited for applications in open field environments. These systems are classified as passive systems since the vapor that results from volatilization at the membrane surface is dispensed by stagnant diffusion and/or random air circulation over the flow control nozzles. The pump is used to move the volatile compound from a storage reservoir to the passive flow control nozzles.

It has been determined that chemoreceptors responsible for repellent response are present on the antennae and other chemosensory organs of mosquitoes and various other arthropod pest species. Moreover, it has been demonstrated that monoterpenoids are capable of activating various chemosensory sensilla on the antennae of various pest species. By decreasing the volatility of the repellent compounds of the present application by means of increasing the molecular weight or polarity of these relatively volatile compounds by synthetic chemistry processes, there is a higher potential for the repellent chemical to remain on surfaces for longer and function as an insect repellent for longer. The novel compounds of the present application can effectively repel pests from a specific target area for longer periods of time than the highly volatile, repellent monoterpenoid compounds they are synthetically derived from.

In carrying out this method of the present application, the term “applying” as used herein includes any suitable method of emitting an effective repellent amount of a plant volatile compound in a target area. The term “target area” as used herein includes any place where the presence of target pests is not desirable, including any type of premises, which can be out-of-doors, such as in gardens, lawns, tents, camping bed nets, camping areas, and so forth, or indoors, such as in barns, garages, commercial buildings, homes, and so forth, or any area where pests are a problem, such as in shipping or storage containers (e.g., bags, boxes, crates, etc.), packing materials, bedding, and so forth. Target area can also include the outer covering of a living being, such as skin, fur, hair, or clothing.

“Applying” includes broadcast or restricted localized spraying of a volatile in or around an area, with or without first micro-encapsulating the volatile, emitting the volatile from one or more controlled-release point-source dispensers in or around an area, and integrating the release of the volatile with an irrigation technique (chemigation). “Applying” can also refer to emitting liquid or solid repellents through use of creams, liquid-based products, powders, and so forth.

A controlled-release point-source dispenser is one type of delivery means for a composition comprising the repellent compound of the present application and a carrier. Such a dispenser includes any suitable device and method for controlling the emission rate of the volatile compound from a concentrated source reservoir of the compounds. For example, and without limitation, suitable dispensers include pads, beads, rods, spirals, or balls comprised of rubber, leather, cotton, wood or wood products, polyethylene, polypropylene or polyvinyl chloride that are impregnated with the volatile compound; micro-capillary tubes open at one end; sealed polyethylene or polypropylene tubes sealed at both ends; laminates comprised of layers of the volatile compound alternated with plastic and cut in various sized flakes or preserved as large ribbons or sheets; permeable or semi-permeable membranes covering a non-permeable container serving as a reservoir for the volatile compounds; large porous beads or sponges; micro-capsules; sealed envelopes or bags made of polyethylene, polypropylene, paper, cardboard, or other permeable substances, metered aerosol systems utilizing pump or pressure technologies to emit aerosolized droplets of the volatiles into the atmosphere, onto plants surfaces or soil, or onto any of the above controlled-release point-source dispensers; and non-aerosol micro-pump technologies that cause metered quantities of the compounds to be dispensed and volatilized by any of the above methods.

A fumigant may also be used in carrying out this aspect of the present application. A “fumigant” as used herein refers to the use of a gas repellent, or a volatile solid or liquid repellent to control pests in storage bins, buildings, ships, rail cars, stored products, organic materials such as soil, foods, animal feed, compost, and so forth, living organisms such as plants, or in any closed areas, i.e., target areas, which are prone to having pests, i.e., pest infestation.

As used herein, the term “repel” means that less time is spent by the pest in a given area, i.e., a target area containing a repellent, than in an available non-target or untreated area (i.e., an area with no repellent). “Repel” can also mean that no time is spent by the pest in the target area. As such, “repelling” a pest includes deterring a pest from remaining in a target area, as well as keeping a pest away from a target area. In some instances, “repel” may include killing a target pest. In some instances, a pest may be “slowed” in behavior and responsiveness after coming in contact with a repellent, such that the presence of the target pest is less of a nuisance to a human or animal in the target area. Slowing a target pest may also allow it to be killed by other means. The total number of pests in an area may be considered to be suppressed or even eliminated due to the repellent compound of the present application. By “suppressed” it is meant to reduce or limit the incidence or severity of a pest infestation or pest activity, even if for a limited period of time.

### EXAMPLES

The examples below are intended to exemplify the practice of embodiments of the disclosure but are by no means intended to limit the scope thereof.

### Example 1 - Monoterpenoid Carbonate Esters as Mosquito Repellents

### Materials and Methods

### General Information

The monoterpenoids used for synthesis were purchased from Sigma-Aldrich, Acros, or TCI, and pyridine and triethylamine were both purchased from TCI. The chloroformates were purchased from Acros, TCI, or Alfa Aesar. All compounds were used as received. All solvents were purchased from Fisher Scientific and used as received. All NMR spectra were obtained at the Iowa State University Chemical Instrumentation Facility using Varian MR 400 MHz and Avance III 600 MHz spectrometers. Chemical shifts are reported relative to the residual solvent peak (CDCl₃: 7.26 ppm for ¹H and 77.16 ppm for ¹³C; DMSO-d₆: 2.50 for ¹H and 39.52 ppm for ¹³C) in ppm (Fulmer et al., “NMR Chemical Shifts of Trace Impurities: Common Laboratory Solvents, Organics, and Gases in Deuterated Solvents Relevant to the Organometallic Chemist,” Organometallics 29:2176-2179 (2010), which is hereby incorporated by reference in its entirety).

### Carbonate Synthesis

In a typical procedure, under an argon atmosphere, monoterpenoid (10 mmol) was dissolved in chloroform (50 mL) along with pyridine (0.949 g, 12 mmol), and the solution was cooled to 0° C. A chloroformate (11 mmol) was added dropwise over two minutes at 0° C. with stirring, and the reaction was then permitted to warm to 22° C., and was stirred at this temperature for 18 hours. The chloroform was removed under vacuum, and the residue was dissolved in ethyl acetate (30 mL) and water (50 mL). The organic layer was isolated, and washed with 1 M hydrochloric acid (20 mL), 1 M sodium hydroxide (20 mL), and brine (20 mL), followed by drying over anhydrous magnesium sulfate. Carbonate esters were all purified using column chromatography using 1:4 methyl t-butyl ether:hexane as eluent unless otherwise noted.

Citronellyl ethyl carbonate (1a) Colorless oil. ¹H NMR (400 MHz, CDCl₃) δ 5.08 (thept, J= 7.1, 1.4 Hz, 1H), 4.13 - 4.03 (m, 4H), 2.08 - 1.87 (m, 2H), 1.78 - 1.68 (m, 1H), 1.68 (s, 1H), 1.59 (d, J = 1.3 Hz, 3H), 1.59 - 1.54 (m, 1H), 1.46 (dtd, J = 13.5, 7.5, 6.0 Hz, 1H), 1.39 - 1.27 (m, 1H), 1.30 (t, J = 7.1 Hz, 3H), 1.18 (dddd, J= 13.5, 9.3, 7.8, 6.0 Hz, 1H), 0.91 (d, J= 6.6 Hz, 3H). ¹³C NMR (101 MHz, CDCl₃) δ 155.4, 131.5, 124.7, 66.5, 63.9, 37.1, 35.7, 29.4, 25.8, 25.5, 19.4, 17.8, 14.4.

Citronellyl methyl carbonate (1b) Colorless oil. ¹H NMR (400 MHz, CDCl₃) δ 5.10 (thept, J= 7.1, 1.4 Hz, 1H), 4.27 - 4.12 (m, 2H), 3.80 (s, 3H), 2.10 - 1.90 (m, 2H), 1.75 (ddd, J= 13.4, 7.3, 5.1 Hz, 1H), 1.70 (q, J= 1.3 Hz, 3H), 1.62 (d, J= 1.3 Hz, 3H), 1.61 - 1.55 (m, 1H), 1.49 (dddd, J= 13.3, 8.0, 7.0, 6.0 Hz, 1H), 1.37 (dddd, J= 13.4, 9.5, 6.5, 5.4 Hz, 1H), 1.20 (dddd, J= 13.6, 9.4, 7.7, 6.0 Hz, 1H), 0.94 (d, J= 6.6 Hz, 3H). ¹³C NMR (101 MHz, CDCl₃) δ 156.0, 131.5, 124.6, 66.8, 54.8, 37.1, 35.6, 29.4, 25.8, 25.5, 19.4, 17.8.

Citronellyl isopropyl carbonate (1c) Isopropyl chloroformate (2 M in toluene) was used instead of neat isopropyl chloroformate. Colorless oil. ¹H NMR (400 MHz, CDCl₃) δ 5.08 (thept, J = 7.1, 1.4 Hz, 1H), 4.87 (hept, J = 6.3 Hz, 1H), 4.23 - 4.07 (m, 2H), 2.08 - 1.87 (m, 2H), 1.77 - 1.68 (m, 1H), 1.68 (s, 1H), 1.59 (d, J= 1.3 Hz, 3H), 1.59 - 1.53 (m, 1H), 1.47 (dddd, J= 13.4, 8.0, 7.2, 6.0 Hz, 1H), 1.35 (dddd, J= 13.6, 7.1, 5.3, 4.1 Hz, 1H), 1.29 (d, J= 6.3 Hz, 7H), 1.18 (dddd, J= 13.6, 9.4, 7.8, 6.0 Hz, 1H), 0.91 (d, J= 6.6 Hz, 3H). ¹³C NMR (101 MHz, CDCl₃) δ 155.0, 131.5, 124.7, 71.8, 66.3, 37.1, 35.7, 29.4, 25.8, 25.5, 21.9, 19.5, 17.8.

Citronellyl isobutyl carbonate (1d) Colorless oil. ¹H NMR (400 MHz, CDCl₃) δ 5.08 (thept, J = 7.1, 1.4 Hz, 1H), 4.24 - 4.09 (m, 2H), 3.91 (d, J = 6.7 Hz, 3H), 2.06 - 1.90 (m, 4H), 1.73 (ddd, J= 13.4, 7.3, 5.1 Hz, 1H), 1.67 (d, J= 1.3 Hz, 3H), 1.59 (d, J= 1.4 Hz, 3H), 1.59 - 1.54 (m, 1H), 1.54 - 1.41 (m, 1H), 1.35 (dddd, J = 13.3, 9.5, 6.4, 5.3 Hz, 1H), 1.18 (dddd, J = 13.6, 9.4, 7.7, 6.0 Hz, 1H), 0.95 (d, J = 6.7 Hz, 6H), 0.91 (d, J = 6.5 Hz, 3H). ¹³C NMR (101 MHz, CDCl₃) δ 155.6, 131.5, 124.6, 74.1, 66.6, 37.1, 35.6, 29.3, 27.9, 25.9, 25.5, 19.5, 19.1, 17.8.

Citronellyl t-butyl carbonate (1e) Citronellol (1.56 g, 10 mmol) and 4-(dimethylamino)pyridine (224 mg, 2 mmol) were dissolved in dichloromethane (50 mL). Di-tert-butyl dicarbonate (2.40 g, 11 mmol) was added in one portion, and the reaction was stirred at 22° C. for 12 hours. The solvent was removed under vacuum, and the reaction was worked up as in the general procedure. The title compound was purified as above to yield a colorless oil (1.98 g, 77%). ¹H NMR (400 MHz, CDCl₃) δ 5.08 (thept, J = 7.1, 1.4 Hz, 1H), 4.24 - 4.09 (m, 2H), 2.07 - 1.91 (m, 4H), 1.77 - 1.67 (m, 1H), 1.69 (d, J = 1.3 Hz, 3H), 1.61 (d, J = 1.4 Hz, 3H), 1.58 - 1.53 (m, 1H), 1.52 (s, 9H), 1.51 - 1.43 (m, 1H), 1.37 (dddd, J= 13.3, 9.6, 6.5, 5.3 Hz, 1H), 1.20 (dddd, J = 13.4, 9.5, 7.7, 6.0 Hz, 1H), 0.91 (d, J = 6.5 Hz, 3H). ¹³C NMR (101 MHz, CDCl₃) δ 155.6, 131.5, 124.7, 81.9, 66.5, 37.1, 35.6, 29.4, 27.9, 25.9, 25.5, 19.5, 17.8.

Citronellyl phenyl carbonate (1f) Colorless oil. ¹H NMR (400 MHz, CDCl₃) δ 7.43 - 7.34 (m, 2H), 7.26 - 7.21 (m, 1H), 7.20 - 7.16 (m, 2H), 5.10 (thept, J= 7.1, 1.4 Hz, 1H), 4.37 - 4.22 (m, 2H), 2.09 - 1.93 (m, 2H), 1.81 (dtd, J = 13.3, 7.3, 4.9 Hz, 1H), 1.72 - 1.63 (m, 1H), 1.69 (q, J= 1.3 Hz, 3H), 1.62 (d, J= 1.4 Hz, 3H), 1.60 - 1.51 (m, 1H), 1.39 (dddd, J= 13.4, 9.4, 6.5, 5.3 Hz, 1H), 1.22 (dddd, J = 13.6, 9.4, 7.7, 6.1 Hz, 1H), 0.96 (d, J = 6.5 Hz, 3H). ¹³C NMR (101 MHz, CDCl₃) δ 153.9, 151.3, 131.6, 129.6, 126.1, 124.6, 121.2, 67.5, 37.1, 35.6, 29.4, 25.9, 25.5, 19.5, 17.8.

Ethyl thymyl carbonate (2a) Colorless oil (1.78 g, 80%). ¹H NMR (400 MHz, CDCl₃) δ 7.20 (d, J = 7.9 Hz, 1H), 7.04 (dd, J = 8.3, 1.1 Hz, 1H), 6.91 (d, J = 1.0 Hz, 1H), 4.32 (q, J = 7.1 Hz, 2H), 3.08 (hept, J = 6.9 Hz, 1H), 2.32 (s, 3H), 1.39 (t, J = 7.1 Hz, 3H), 1.21 (d, J = 6.9 Hz, 6H). ¹³C NMR (101 MHz, CDCl₃) δ 154.1, 148.4, 137.2, 136.8, 127.5, 126.7, 122.5, 64.9, 27.1, 23.2, 21.0, 14.4.

Isobutyl thymyl carbonate (2d) Triethylamine (1.21 g, 12 mmol) used instead of pyridine. Colorless oil. ¹H NMR (400 MHz, CDCl₃) δ 7.19 (d, J= 7.9 Hz, 1H), 7.04 (dd, J = 7.7, 1.8 Hz, 1H), 6.91 (d, J = 2.1 Hz, 1H), 4.05 (d, J = 6.7 Hz, 1H), 3.08 (hept, J = 6.9 Hz, 1H), 2.32 (s, 1H), 2.06 (thept, J = 6.9, 6.7 Hz, 1H), 1.21 (d, J = 6.9 Hz, 3H), 1.01 (d, J = 6.7 Hz, 3H). ¹³C NMR (101 MHz, CDCl₃) δ 194.3, 154.3, 148.4, 137.2, 136.8, 127.5, 126.7, 122.5, 74.8, 28.0, 27.1, 23.2, 21.0, 19.0.

Ethyl isopulegyl carbonate (3a) Colorless oil. ¹H NMR (400 MHz, CDCl₃) δ 4.773 (s, 1H), 4.769 (s, 1H), 4.62 (td, J = 10.9, 4.4 Hz, 1H), 4.20 - 4.08 (m, 2H), 2.19 - 2.02 (m, 2H), 1.77 -1.65 (m, 2H) 1.69 (s, 1H), 1.54 (dtdt, J = 16.4, 10.1, 6.8, 3.2 Hz, 1H), 1.37 (qd, J = 14.5, 13.8, 4.1 Hz, 1H), 1.27 (t, J = 7.2 Hz, 3H), 1.09 (td, J = 12.2, 11.0 Hz, 1H), 0.94 (d, J = 6.6 Hz, 3H). ¹³C NMR (101 MHz, CDCl₃) δ 154.8, 145.8, 112.0, 77.4, 63.6, 50.5, 40.3, 34.0, 31.4, 30.4, 22.0, 19.6, 14.2.

Ethyl geranyl carbonate (4a) Colorless oil. ¹H NMR (400 MHz, CDCl₃) δ 5.37 (th, J = 7.1, 1.3 Hz, 1H), 5.07 (thept, J = 6.8, 1.5 Hz, 1H), 4.64 (dd, J = 7.2, 0.9 Hz, 2H), 4.19 (q, J = 7.1 Hz, 2H), 2.17 - 1.97 (m, 4H), 1.71 (d, J = 1.4 Hz, 3H), 1.67 (d, J = 1.4 Hz, 3H), 1.59 (d, J= 1.4 Hz, 3H), 1.30 (t, J= 7.1 Hz, 3H). ¹³C NMR (101 MHz, CDCl₃) δ 155.4, 143.2, 132.0, 123.8, 117.9, 64.6, 64.0, 39.6, 26.4, 25.8, 17.8, 16.6, 14.4.

Ethyl menthyl carbonate (5a) Colorless oil. ¹H NMR (400 MHz, CDCl₃) δ 4.50 (td, J = 10.9, 4.5 Hz, 1H), 4.18 (qd, J = 7.1, 2.3 Hz, 2H), 2.07 (dtd, J = 12.0, 4.1, 1.8 Hz, 1H), 1.97 (heptd, J = 7.0, 2.7 Hz, 1H), 1.73 - 1.62 (m, 2H), 1.48 (dddd, J = 15.2, 8.6, 6.5, 3.3 Hz, 0H), 1.40 (ddt, J= 12.4, 10.8, 3.1 Hz, 1H), 1.30 (t, J = 7.1 Hz, 3H), 1.12 - 0.98 (m, 2H), 0.90 (t, J= 7.0 Hz, 6H), 0.78 (d, J= 7.0 Hz, 3H). ¹³C NMR (101 MHz, CDCl₃) δ 155.0, 78.2, 63.8, 47.1, 40.9, 34.2, 31.5, 26.1, 23.4, 22.1, 20.9, 16.3, 14.4.

Ethyl perillyl carbonate (6a) Colorless oil. ¹H NMR (400 MHz, CDCl₃) δ 5.80 (tt, J = 2.9, 1.3 Hz, 1H), 4.75 - 4.67 (m, 2H), 4.55 - 4.45 (m, 2H), 4.19 (q, J= 7.1 Hz, 2H), 2.21 - 2.06 (m, 4H), 2.02 - 1.90 (m, 1H), 1.84 (ddq, J= 10.6, 4.1, 2.2 Hz, 1H), 1.73 (d, J= 1.2 Hz, 3H), 1.55 - 1.40 (m, 1H), 1.30 (t, J = 7.2 Hz, 3H). ¹³C NMR (101 MHz, CDCl₃) δ 155.4, 149.7, 132.3, 126.8, 108.9, 71.9, 64.1, 40.8, 30.6, 27.4, 26.4, 20.9, 14.4.

Bornyl ethyl carbonate (7a) Colorless oil. ¹H NMR (400 MHz, CDCl₃) δ 4.80 (ddd, J = 9.9, 3.5, 2.1 Hz, 1H), 4.18 (q, J = 7.1 Hz, 2H), 2.37 (dddd, J = 13.6, 9.9, 4.7, 3.3 Hz, 1H), 2.01 - 1.89 (m, 1H), 1.82 - 1.70 (m, 1H), 1.68 (t, J = 4.5 Hz, 1H), 1.32 (t, J = 7.1 Hz, 3H), 1.30 - 1.21 (m, 1H), 1.09 (dd, J = 13.8, 3.5 Hz, 1H), 0.90 (s, 3H), 0.88 (s, 3H), 0.87 (s, 3H). ¹³C NMR (101 MHz, CDCl₃) δ 155.7, 83.8, 63.9, 49.0, 48.1, 44.9, 36.6, 28.1, 27.0, 19.9, 19.0, 14.5, 13.6.

### Mosquito Repellency Testing

The static air chamber used for the repellency assay has been described previously (Klimavicz et al., “Monoterpenoid Isovalerate Esters as Long-lasting Spatial Mosquito Repellents,” In Advances in the Biorational Control of Medicaland Veterinary Pests, American Chemical Society: 2018; Vol. 1289, pp 205-217; Peterson et al., “Identification of Components of Osage Orange Fruit (Maclura pomifera) and Their Repellency to German Cockroaches,” J. Essent. Oil Res. 14:233-236 (2002), which are hereby incorporated by reference in their entirety). For each assay run, a 90-mm Whatman No. 1 filter paper was treated with 1 mL acetone solution containing 0.5% w/v of a carbonate ester. The filter paper was supported upon several pins during the addition of the acetone solution to diminish wicking of the solution away from the filter paper, and the solution was added over approximately thirty seconds to allow acetone to evaporate during the addition to prevent dripping. After the addition of the solution was complete, remaining acetone was permitted to evaporate for 10 minutes before testing.

A 600-mm-long clear glass cylinder with an inner diameter of 85 mm and a single hole (20 mm diameter) halfway along the side of the cylinder was used for the repellency chamber. One end of the chamber was capped with a glass petri dish (inner diameter 90 mm) containing an untreated 90-mm Whatman No. 1 filter paper, while the other end was likewise covered with a similar petri dish containing the treated filter paper. Immediately after capping both ends of the chamber, 20 non-blood-fed female Culex pipiens mosquitoes were anesthetized with carbon dioxide, and placed into the repellency chamber through the hole in the side of the cylinder via a funnel. The hole was sealed, and the number of mosquitoes on the treated and untreated halves were recorded at 15, 30, 60, 90, 120, and 150 minutes after introduction of the mosquitoes to the chamber.

### Data Analysis

All statistical analysis was performed in R. To determine repellency of a given compound, the number of repelled mosquitoes and the total number of mosquitos the chamber for each trial at a given time point were fit to a beta-binomial distribution to account for overdispersion in the estimated repellency. Because percentage repellency is bound between -100% (completely attracting) and 100% (completely repelling), percentage repellency shown with error bars representing 50% confidence intervals determined from a non-chance-corrected beta-binomial fit to the data, using an inverse link function equal to the psychometric function for the duo-trio test. Statistical significance was determined by nonoverlap of these confidence intervals. Estimates of the proportion of mosquitoes repelled in the chamber was then transformed to percentage repellency, which is expressed as

$${\%}_{\text{r}} = 100 \% ⋅ \frac{{\text{n}}_{\text{t}} - {\text{n}}_{\text{u}}}{{\text{n}}_{\text{t}} + {\text{n}}_{\text{u}}} .$$

### Results and Discussion

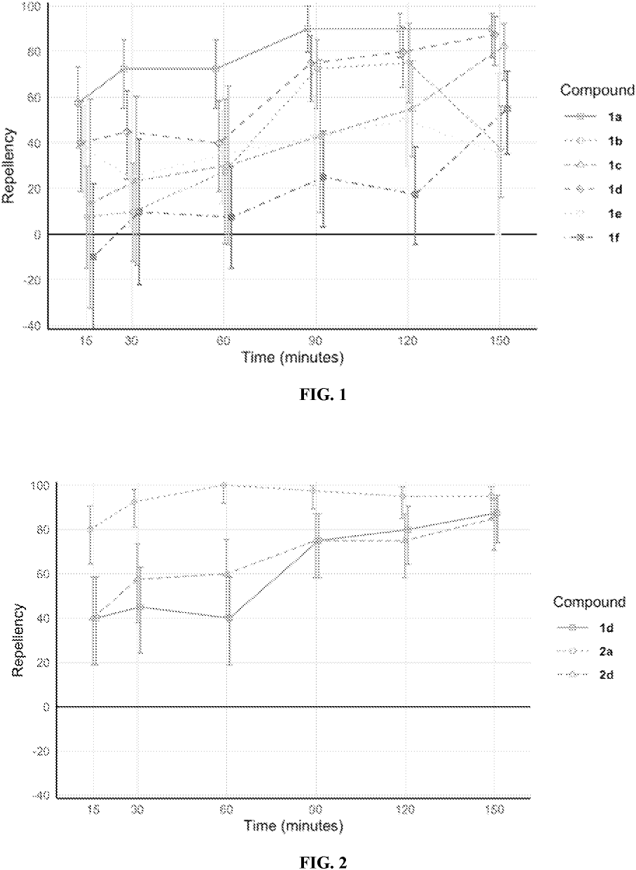

A series of citronellyl carbonates was first made, because amongst a series of isovalerate esters that have been previously tested as short- and long-term mosquito repellents, citronellyl isovalerate provided a moderate response, allowing distinctions to be made between different levels of efficacy. Using commercially available ethyl chloroformate, citronellyl ethyl carbonate (1a) was synthesized as the first carbonate, since this choice of alkyl group produced a compound with a molecular weight most similar to sesquiterpenoid alcohols, which has previously been successful as a strategy in developing long-lasting spatial mosquito repellents. Using other chloroformates and citronellol, the corresponding methyl, isopropyl, isobutyl, and phenyl carbonates (1b, 1c, 1d, and 1f, respectively) were also synthesized. Citronellyl tert-butyl carbonate (1e) was also produced using di-tert-butyl dicarbonate. The short-term repellency trends of these compounds are shown in FIG. 1. Of the compounds in this series, the somewhat bulky 1f produced the weakest repellent effect, and was statistically significantly poorer than many of the other carbonates, all of which had lower molecular weight than 1f. Repellent 1a was numerically superior out of all the citronellyl carbonates, while the methyl, isopropyl, and isobutyl derivatives were numerically, but not statistically, less repellent than 1a.

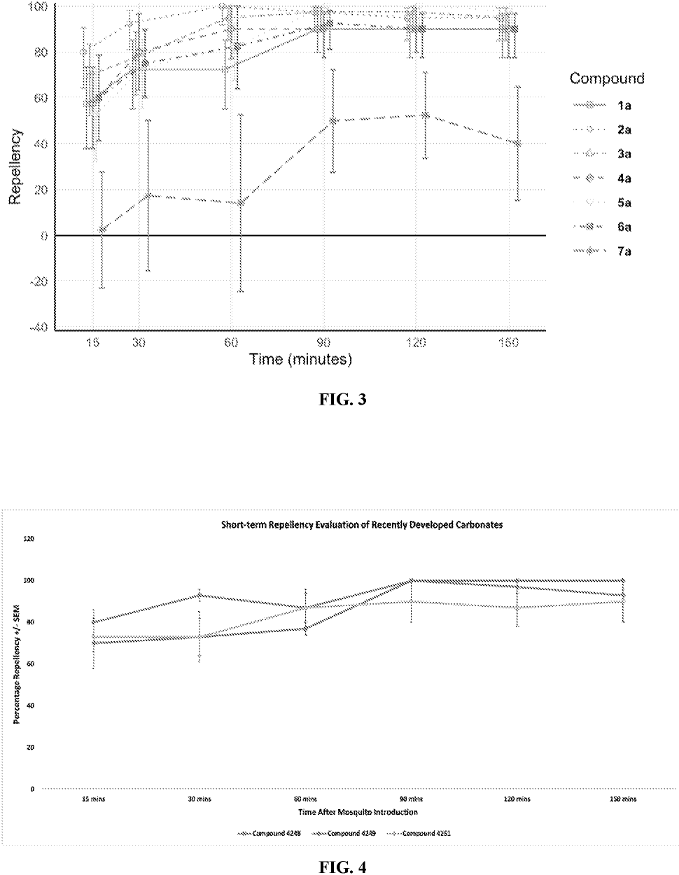

Given the success of 1a, it was then elected to synthesize other ethyl monoterpenoid carbonates to explore trends in short-term repellency; these results are shown in FIG. 3. A selection of structurally diverse monoterpenoids was chosen for synthesis and testing and comparison to 1a. The thymyl, isopulegyl, geranyl, menthyl, and perillyl ethyl carbonates (compounds 2a, 2b, 2c, 2d, and 2e, respectively) were readily synthesized from their corresponding monoterpenoid alcohols, and showed remarkably good short-term repellency, with all of 1a and 2a-e repelling 90% or more of the C. pipiens after the 90-minute time point. Bornyl ethyl carbonate (7a), again easily synthesized from borneol, was the one exception to the high repellency of these ethyl carbonates, and only modest repellency was observed, significantly lower than that seen for the other monoterpenoid ethyl carbonates. It was also attempted to synthesize the ethyl carbonates of linalool and α-terpineol; however, the reaction was unsuccessful with these sterically-hindered tertiary monoterpenoids.

Isobutyl thymyl carbonate (2d) was then produced to determine if other monoterpenoid isobutyl carbonates are similar in repellency to 1d. The results of this examination are shown in FIG. 2. Ethyl thymyl carbonate was an excellent short-term repellent, and achieves high repellency in the static air chamber much more quickly than does the isobutyl analog, as 2d more closely follows the pattern of the corresponding citronellol derivative 1d. This slower onset of high repellency may be a result of the higher molecular weight, since the final estimated repellency was approximately the same for both 2a and 2d.

### Conclusions

Volatile monoterpenoid carbonates represent a new class of spatial insect repellents, and many of these compounds are excellent spatial repellents against Culex pipiens. In particular, most of the monoterpenoid ethyl carbonates screened in this study repelled 90% or more of the mosquitoes in our bioassay.

### Example 2 - Additional Repellency Data

An array of older and newly synthesized carbonates was chosen and tested for repellency activity. Six compounds were screened according to long-term repellency assays and three compounds were screened according to short-term repellency assays. These assays were all conducted using Culex pipiens mosquitoes. The compounds tested are illustrated in Table 1.

TABLE 1

Compounds Tested in Repellency Tests

| Compound Number | Compound Identity |
|---|---|
| 3170 | Ethyl thymyl carbonate |
| 3173 | Ethyl isopulegyl carbonate |
| 3202 | Ethyl geranyl carbonate |
| 3204 | Ethyl perillyl carbonate |
| 3205 | Isobutyl thymyl carbonate |
| 3207 | Citronellyl isobutyl carbonate |
| 4248 | Thymyl isopropyl carbonate |
| 4249 | Thymyl methyl carbonate |
| 4251 | Verbenyl ethyl carbonate |

The results shown in FIG. 4 indicate that the three compounds screened were all quite effective at repelling mosquitoes in the short-term assays. It is also worth noting that in the trials using Compound 4249, significant mortality was seen in the chamber. Very minimal amounts of mortality were seen in Compound 4251. These findings indicate that the compounds are quite repellent and potentially toxic to the mosquitoes. These compounds may be quite effective at lesser concentrations when used as spatial repellents.

The results shown in FIG. 5 indicate that the six compounds screened for their long-term repellent properties displayed various amounts of repellency. Some of these compounds were able to achieve near 100% repellency over the course of this assay. Compounds such as Compound 3173 was not able to repel mosquitoes very well at all over the course of this assay. These results are very similar to previous data seen when testing these compounds in the short-term assays. These results suggest that this carbonate class of compounds could serve as quite effective spatial repellents.

### Example 3 - Additional Repellency Data

An array of carbonates was chosen and tested for repellency activity. Some compounds were screened according to long-term repellency assays and some compounds were screened according to short-term repellency assays. These assays were conducted using Culex pipiens mosquitoes or Aedes aegypti mosquitoes.

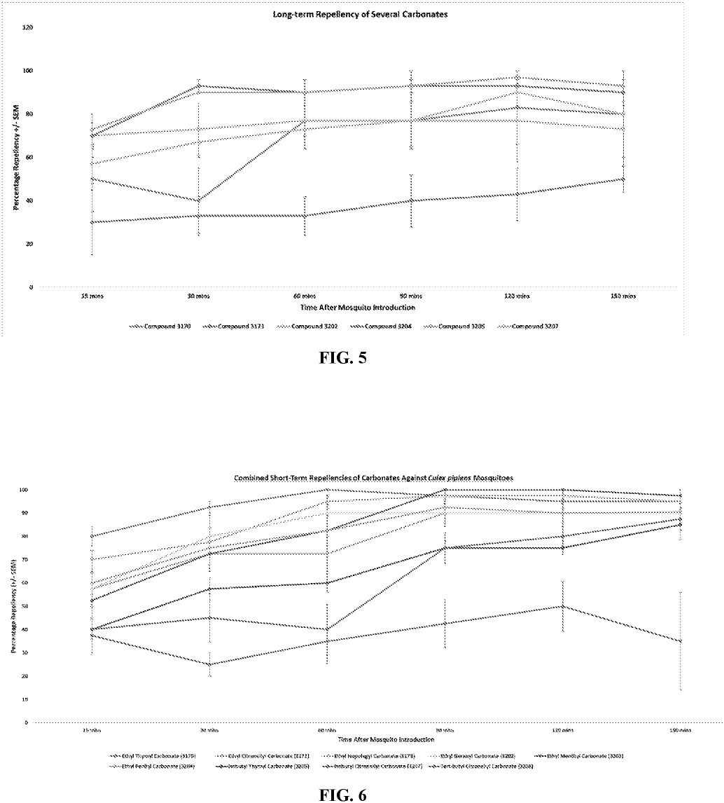

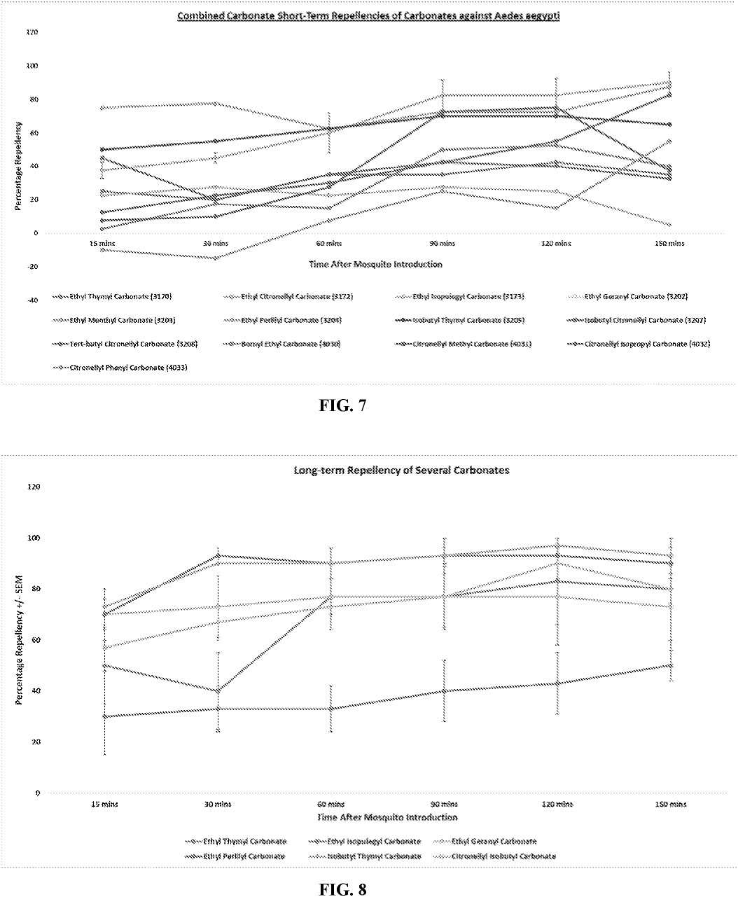

The results shown in FIG. 6 and FIG. 7 indicate the efficacy of the compounds at repelling mosquitoes in the short-term assays. These findings indicate that the compounds are quite repellent and potentially toxic to the mosquitoes. These compounds may be quite effective at lesser concentrations when used as spatial repellents.

The results shown in FIG. 8 indicate that compounds screened for their long-term repellent properties displayed various amounts of repellency. Some of these compounds were able to achieve near 100% repellency over the course of this assay. Other compounds were not able to repel mosquitoes very well at all over the course of this assay. These results are very similar to previous data seen when testing these compounds in the short-term assays. These results suggest that this carbonate class of compounds could serve as quite effective spatial repellents. As a class, the carbonates presented in the present application are more effective repellents than previously tested monoterpenoid ester repellents.

Although preferred embodiments have been depicted and described in detail herein, it will be apparent to those skilled in the relevant art that various modifications, additions, substitutions, and the like can be made without departing from the spirit of the application and these are therefore considered to be within the scope of the application as defined in the claims which follow.

What is claimed is:

1. A compound of formula (I) having the following structure: 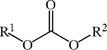
or a stereoisomer, salt, oxide, or solvate thereof, wherein
R¹ is a monoterpenoid or phenylpropanoid moiety;
R² is selected from C₁-C₁₀ alkyl optionally substituted with halogen, C₂-C₁₀ alkenyl, phenyl, and —(CH₂)ₙ—phenyl; and
n is an integer from 0-3.

2. The compound of claim 1, wherein R¹ is a monoterpenoid moiety.

3. The compound of claim 2, wherein the monoterpenoid moiety is selected from the group consisting of 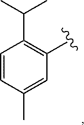 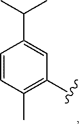 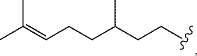 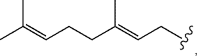 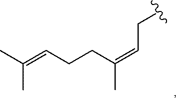 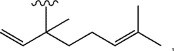 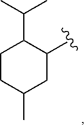 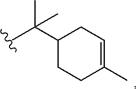 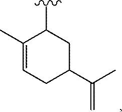 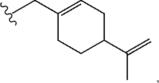 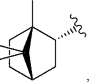 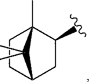 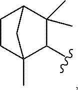 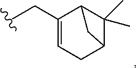 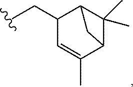 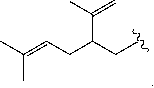 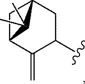 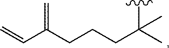 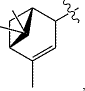 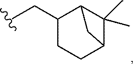 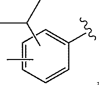 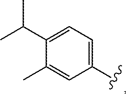 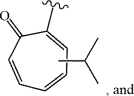 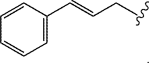
.

4. The compound of claim 1, wherein R¹ is a phenylpropanoid moiety.

5. The compound of claim 4, wherein the phenylpropanoid moiety is selected from the group consisting of 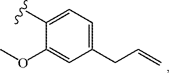 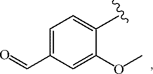 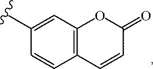 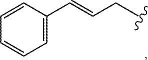 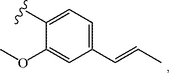 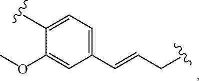 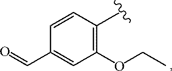 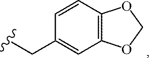 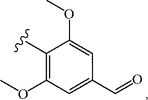 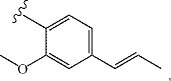 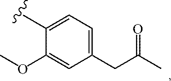 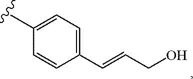 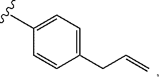 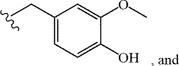 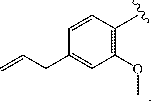
.

6. The compound of any one of claims 1-5, wherein R² is C₁-C₁₀ alkyl.

7. The compound of claim 6, selected from the group consisting of 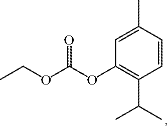 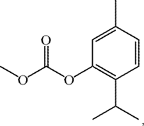 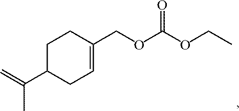 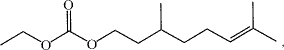 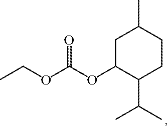 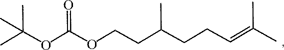 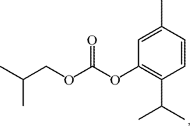 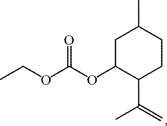 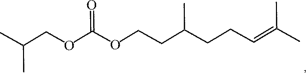 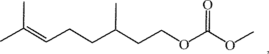 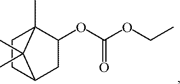 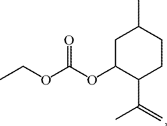 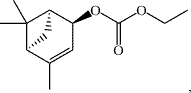 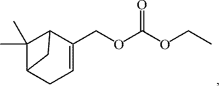 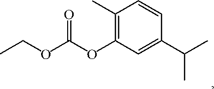 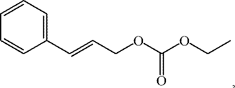 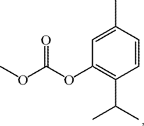 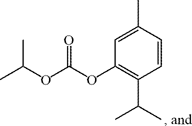 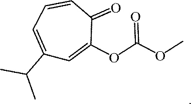
.

8. The compound of any one of claims 1-5, wherein R² is C₁-C₁₀ alkyl substituted with halogen.

9. The compound of claim 8, having the following structure: 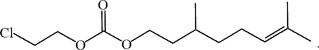
.

10. The compound of any one of claims 1-5, wherein R² is C₂-C₁₀ alkenyl.

11. The compound of claim 10, having the following structure: 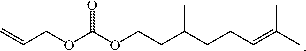
.

12. The compound of any one of claims 1-5, wherein R² is phenyl.

13. The compound of claim 12, having the structure: 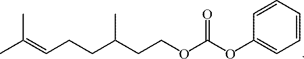
.

14. The compound of any one of claims 1-5, wherein R² is —(CH₂)ₙ—phenyl.

15. The compound of claim 14, having the structure: 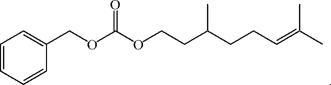
.

16. A composition comprising:
a carrier; and
a compound of formula (I) having the following structure: 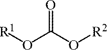
or a stereoisomer, salt, oxide, or solvate thereof, wherein
R¹ is a monoterpenoid or phenylpropanoid moiety;
R² is selected from C₁-C₁₀ alkyl optionally substituted with halogen, C₂–C₁₀ alkenyl, phenyl, and —(CH₂)ₙ—phenyl; and
n is an integer from 0-3.

17. The composition of claim 16, wherein the compound is in a substantially pure form.

18. The composition of claim 16 or claim 17, wherein the compound is a single enantiomer or diastereomer.

19. The composition of claim 16 or claim 17, wherein the compound is in a racemic or diastereomeric mixture.

20. The composition of any one of claims 16-19, wherein the carrier is selected from a solid, liquid, and gas.

21. The composition of any one of claims 16-20, wherein the composition is in the form of a lotion, spray, or cream.

22. The composition of any one of claims 16-21 further comprising:
a fragrance, perfume, or cologne.

23. A method of repelling a pest, said method comprising:
applying to a target area a composition comprising a compound of formula (I) having the following structure: 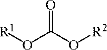
or a stereoisomer, salt, oxide, or solvate thereof, wherein
R¹ is a monoterpenoid or phenylpropanoid moiety;
R² is selected from C₁-C₁₀ alkyl optionally substituted with halogen, C₂-C₁₀ alkenyl, phenyl, and —(CH₂)ₙ—phenyl; and
n is an integer from 0-3;
wherein said applying is carried out under conditions effective to repel a pest.

24. The method of claim 23, wherein said applying is carried out with a vapor delivery system.

25. The method of claim 23 or claim 24, wherein the pest is selected from the group consisting of blood-sucking insects, biting insects, cockroaches, mosquitoes, blackfly, fleas, house flies, barn fly, face fly, bush fly, deer fly, horse fly, gnats, beetle, beer bug, louse, bed bug, earwig, ant, aphid, spruce bud worm, corn borer, sand flea, tsetse fly, assassin bug, biting flies, sand fly, stored grain pests, clothes moths, ticks, mites, spiders, phytophagous pests, and hematophagous pests.
